SEQUENCE LISTING (8 sequences)

SEQ ID NO: 1 - DNA, 20 bp, Artificial
  primer DNA based on EBER1
aaaacatgcggaccaccagc

SEQ ID NO: 2 - DNA, 20 bp, Artificial
  primer DNA based on EBER1
aggacctacgctgccctaga

SEQ ID NO: 3 - DNA, 26 bp, Artificial
  primer DNA based on Beta2-microglobulin
tacatgtctcgatcccacttaactat

SEQ ID NO: 4 - DNA, 23 bp, Artificial
  primer DNA based on Beta2-microglobulin
agcgtactccaaagattcaggtt

SEQ ID NO: 5 - DNA, 24 bp, Artificial
  outer primer DNA based on BARTs
tgagggaaataaccaggatcacca

SEQ ID NO: 6 - DNA, 21 bp, Artificial
  outer primer DNA based on BARTs
gcttctcctcggacatccagt

SEQ ID NO: 7 - DNA, 27 bp, Artificial
  inner primer DNA based on BARTs
tgaagaaggagatgaaaccagagacca

SEQ ID NO: 8 - DNA, 20 bp, Artificial
  inner primer DNA based on BARTs
gacgaacagcgtgcctccaa

(19) United States
(12) Patent Application Publication
(10) Pub. No.: US 2009/0047657 A1
(43) Pub. Date: Feb. 19, 2009

(54) Detection Method For Latent Viral Infections and Its Kit For Examination

(76) Inventors: Keiji Iwatsuki, Okayama (JP); Takenobu Yamamoto, Okayama (JP)

Correspondence Address: KILYK & BOWERSOX, P.L.L.C., 400 HOLIDAY COURT, SUITE 102, WARRENTON, VA, 20186, US

(21) Appl. No.: 12/065,679

(22) PCT Filed: Sep. 8, 2006
(86) PCT No.: PCT/JP2006/317851

§ 371 Date: Mar. 18, 2008

(30) Foreign Application Priority Data
Sep. 9, 2005 (JP) 2005-261917

(51) Int. Cl. C12Q 1/70

(52) U.S. Cl. 435/5

(57) ABSTRACT

The subject of the present invention is to provide a detection method for latent viral infections by detecting a gene product related to latent infections without going through an invasive procedure involving pain and bleeding, caused by skin biopsy and blood collection. Further, the subject of the present invention is to provide a kit for examination using in the detection of latent viral infections described above. As crusts and scales in a lesion contain large amounts of virus-infected cells which are in a state of dry necrosis, the method collects crusts and/or scales for a test sample and detects a gene product related to latent infections which may be present in the test sample. The kit for examination comprises (1) antisense oligonucleotide for reverse transcription of a gene product related to latent viral infections, (2) a primer set for amplifying a gene product related to latent viral infections and (3) a primer set for amplifying a housekeeping gene.

### TECHNICAL FIELD

The present invention relates to a detection method for latent viral infections by detecting a gene product related to latent infections generated by latent viral infections. In particular, the present invention relates to a detection method for latent viral infections by detecting a gene product related to latent infections generated by latent infections of virus which can be present in crusts (scabs) and scales appeared on the skin. Further, the present invention relates to a kit for examination using in these detection methods.

This application claims the priority of Japanese Patent Application No. 2005-261917, which is incorporated herein by reference.

### BACKGROUND ART

Epstein-Barr virus (hereinafter referred to as simply “EB virus”), a member of Herpesviridae family, was discovered nearly 40 years ago in Burkitt's Lymphoma which is among one of tumors often found in African children. EB virus usually proliferates via saliva, infects B-cell, which is one type of the human lymphocytes, as the main target, and then latently and persistently spreads in the internal body. Infectious mononucleosis is referred to a case where initial infections of B-cell with the EB virus develop(s) symptoms such as fever and hepatosplenomegaly. However, even infected, most initial infections are symptomless in infants, whose immune system are poorly developed (inapparent infections), so diagnosis of infectious mononucleosis is often made in the case of initial infections in a part of infants, adolescents and adults.

Typical diseases caused by the EB virus include infectious mononucleosis and EB virus-related hemophagocytic syndrome in cases of acute infections, and Burkitt's lymphoma, nasopharyngeal carcinoma and gastric cancer in malignant cases. The further included is chronic active EB virus infections.

Chronic active EB virus infections are induced by the infections mostly of T-cells and NK-cells except B-cells with EB virus. Symptoms include, for example, fever, hepatomegaly, splenomegaly and lymphadenopathy, and if EB virus infects NK-cells, symptoms such as hypersensitivity to mosquito bites (mosquito allergy) may appear. As a symptom of hypersensitivity to mosquito bites, there shown in severe cases are fever, flare and swelling in the area of mosquito bites which may develop to the size of 10-20 cm or more, followed by the formation of blisters, and growing to ulcers in the size of 1 to 2 cm. Further, chronic diarrhea accompanied by malabsorption; interstitial pneumonia; cardiovascular disorders such as myocarditis and coronary artery aneurysm; neurologic symptoms such as uveitis, encephalitis, myelitis and peripheral neuritis are comparatively and frequently observed. The prognosis is extremely poor and finally multiple organ failures such as cardiac, hepatic and renal failures and malignant diseases such as malignant lymphoma and leukemia may sometimes appear in several years.

Various detection methods for the virus have been examined and many methods reported. A method by isolating virus from body fluids (such as blood and saliva), tissues or cells and identifying the virus, or an immunological method by measuring, for example, viral capsid antigen (VCA) or intranuclear antigen (EBNA) in the serum were reported. However, there are problems, for example, in that diagnosis of viral isolation may be time-consuming until the result obtained, and that immunological methods may detect reactions nonspecific to antibodies, and that high sensitivity is difficult to obtain.

Later, as a direct method for detecting virus in tissue and body sample from patient, such methods were reported as, concerning EB virus, a detection method by reacting a viral antigen present in a sample with an antibody labeled with fluorescence, a PCR method comprising performing skin biopsy, extracting DNA from the obtained tissue and amplifying the DNA (Patent Document No. 1), a real-time PCR method (Patent Document No. 2) and a method of amplifying nucleic acid such as RT-PCR method comprising preparing cDNA from RNA using reverse transcriptase and amplifying the DNA. Further, a method for detecting an EB virus-related product in skin sections by in situ hybridization (Patent Document No. 3), a method for detecting a fragment of intracellular EB virus by Southern blotting and the like have been reported. In addition, an oligonucleotide for detecting ES virus applicable to nucleic acid amplification and in situ hybridization (Patent Document No. 4) was reported.

In any event, the methods described above collect samples by an invasive procedure involving pain and bleeding, caused by skin biopsy and blood collection, so the examination causes deep suffering especially on child patients.

### [Patent Document No. 1] Japanese Patent No. 3360737-B

[Patent Document No. 2] Japanese Published Patent Application No. Hei 11-137300-A

### [Patent Document No. 3] Japanese Published Patent Application No. 2003-24077-A

[Patent Document No. 4] Published Japanese translation of PCT Application No. 2002-505122-A

### DISCLOSURE OF THE INVENTION

### Problems to be Solved by the Invention

The subject of the present invention is to provide a detection method for latent viral infections by detecting a gene product related to latent infections without going through an invasive procedure involving pain and bleeding, caused by skin biopsy and blood collection. Further, the subject of the present invention is to provide a kit for examination using in the detection of latent viral infections described above.

### Means for Solving Problem

The present inventors have strenuously studied to solve the matters described above and found that crusts and scales in a lesion contain large amounts of virus-infected cells, so we focused on that those cells are in a state of dry necrosis and verified that collecting crust and scale and using them as a test sample allows a gene product related to latent infections in a virus-infected cell to be extracted in a stable condition, thereby completing the present invention.

Therefore, the present invention consists of the following:

2. A detection method for latent viral infections, wherein a gene product related to latent infections which may be present in test sample are detected, comprising the following steps:
1) collecting crust and/or scale formed on the skin for a test sample,
2) extracting RNA which is a gene product related to latent viral infections from the test sample,
3) amplifying nucleic acid based on the extracted RNA, and
4) detecting a product generated by latent viral infections in the amplified nucleic acid.
3. The detection method according to the preceding aspect 2, wherein the virus is EB virus (Epstein-Barr virus) or KSHV (Kaposi's sarcoma-associated herpes virus).
4. The detection method according to the preceding aspect 3, wherein the gene product related to latent viral infections is RNA which may be present in the nucleus or cytoplasm of an infected cell.
5. The detection method according to the preceding aspect 4, wherein RNA which may be present in the nucleus or cytoplasm of an infected cell is EB virus-encoded small RNA (EBER) derived from ES virus and/or BamHI A rightward transcripts (BARTs (BARFO)).
6. The detection method according to the preceding aspect 4, wherein the step of amplifying nucleic acid comprises a step of simultaneously amplifying EBER-derived and β2-microglobulin-derived nucleic acids under a same amplification condition.
13. The detection method according to the preceding aspect 6, wherein the oligonucleotides shown as SEQ ID NO: 2 (5′-AGGACCTACGCTGCCCTAGA-3′) and SEQ ID NO: 1 (5′-AAAACATGCGGACCACCAG C-31) in the sequence listing are used as a primer set for amplifying EBER-derived nucleic acid, and the oligonucleotides shown as SEQ ID NO: 3 (5′-TACATGT CTCGATCCCACTTAACTAT-3′) and SEQ ID NO: 4 (5′-AGCGTACTCCAAAGATTCAGGTT-3′) in the sequence listing are used as a primer set for amplifying β2-microglobulin-derived nucleic acid.
14. The detection method according to the preceding aspect 6 or 13, wherein the step of amplifying nucleic acid further comprises a step of amplifying nucleic acid derived from BARTs (BARF0).
15. The detection method according to the preceding aspect 14, the oligonucleotides shown as both SEQ ID NO: 5 (5′-TGAGGGAAATAACCAGGATCACCA-3′) and SEQ ID NO: 6 (5′-GCTTCTCC TCGGACATCCAGT-3′) and both SEQ ID NO: 7 (5′-TGAAGAAGGAGATGAAACCAGAGACCA-3′) and SEQ ID NO: 8 (5′-GACGAACAGCGTGCCTCCAA-3′) in the sequence listing are used as primer sets for amplifying nucleic acid derived from BARTs (BARF0).
16. The detection method according to any one of the preceding aspects 6, 13 to 15, wherein the step of amplifying nucleic acid is performed by polymerase chain reaction (PCR) operation and a final concentration of MgCl₂ is 1.5±0.2 mM per 25 μl of cDNA mixture for PCR operation.
17. The detection method according to the preceding aspect 16, wherein annealing is performed at temperature in the range of 62±2° C. and/or 64±2° C. in the PCR operation.
18. A kit for examination using in the detection method according to any one of the preceding aspects 2 to 6, 13 to 15, comprising at least the following 1) to 3):
1) antisense oligonucleotide for reverse transcription of a gene product related to latent viral infections,
2) a primer set for amplifying a gene product related to latent viral infections,
3) a primer set for amplifying a housekeeping gene.
19. The kit for examination according to the preceding aspect 18, wherein the antisense oligonucleotide and/or the primer set in said 1) to 3) is the following: 1) the oligonucleotide shown as SEQ ID NO: 1 (5′-AAAACATGCGGACCACCAGC-3′) in the sequence listing,
2) a primer set for amplifying EBER-derived nucleic acid, wherein the primer set is made up by combining the oligonucleotides shown as SEQ ID NO: 2 (5′-AGGACCTACGCTGCCCTAGA-3T) and SEQ ID NO: 1 (5′-AAAACATGCGGACCACCAGC-3′) in the sequence listing,
3) a primer set for amplifying β2-microglobulin-derived nucleic acid, wherein the primer set is made up by combining the oligonucleotides shown as SEQ ID NO: 3 (5′-TACATGTCTCGATCCCACTTAACTAT-3′) and SEQ ID NO: 4 (5′-AGCGTACTCCAAAGATTCAGGTT-3′) in the sequence listing.
20. The kit for examination according to the preceding aspect 19, further comprising a primer set for amplifying nucleic acid derived from BARTs (BARF0), wherein the primer set is made up by combining the oligonucleotides shown as both SEQ ID NO: 5 (5′-TGAGGGAAATAACCAGGATCACCA-3′) and SEQ ID NO: 6 (5′-GCTTCTCCTCGGACATCCAGT-3′) and both SEQ ID NO: 7 (5′-TGAAGAAGGAGATGAAACCAGAGACCA-3′) and SEQ ID NO: 8 (5′-GACGAACAGCGTGCCTCCAA-3′) in the sequence listing.

### EFFECTS OF INVENTION

The method of the present invention which comprises collecting crusts and scales in a lesion and using them as a test sample can obtain a test sample without pain or invasion. In particular, as diseases caused by latent infections of EB viruses, such as hydroa vacciniforme, hypersensitivity to mosquito bites and chronic active EB virus infections are often found in children, clinical examinations involving pain and invasion should be avoided as much as possible.

### BRIEF DESCRIPTION OF THE DRAWINGS

FIG. 1 shows the result of testing for EB virus-infected cell using 2% agarose gel. (Example 1)

FIG. 2 shows the result of detection of EBER1 and BARTs (BARF0) using 2% agarose gel. (Example 2)

### EXPLANATION OF LETTERS OR NUMERALS

1 to 7: Skin disease cases related to EB virus
A to D: Skin disease cases unrelated to EB virus

### DESCRIPTION OF THE PREFERRED EMBODIMENT

The detection method for latent viral infections of the present invention comprises a step of collecting crusts and/or scales formed on the skin for a test sample.

Virus for performing the examination for latent viral infections of the present invention is not limited in particular as long as it can cause latent infections.

The detection method for latent viral infections of the present invention is not limited to an examination for humans, but the meaning may be found in reducing the suffering which human has at the time of examination in particular, and therefore, in that sense, the method will preferably be applied to examinations for virus infectious to humans.

There are eight kinds of herpes viruses which are known to be infectious to human. Among them, three kinds, herpes simplex virus types 1 and 2 and varicella-zoster virus, form blisters on the skin after infections. EB virus (Epstein-Barr virus) induces infectious mononucleosis, while cytomegalovirus causes not only severe infections in neonates and people with impaired immune function but also symptoms similar to infectious mononucleosis in people having normal immune function.

Human herpes virus types 6 and 7 cause a child disease known as exanthem subitum. It is pointed out that KSHV (Kaposi's sarcoma-associated herpes virus, Kaposi's sarcoma-related herpes virus), which is human herpes virus type 8, is related to a cancer called Kaposi's sarcoma which is shown in AIDS patients.

As a virus for the present invention, herpes viruses are preferred in particular as an example. Among herpes viruses, the present invention may be applied to EB virus or KSHV for which latent infections can be a problem in particular.

Viral infections sometimes follows a course: blisters appearing on the skin and in the lining and then naturally broken, and crusts formed in several days and cured, for example. In addition, hypersensitivity to mosquito bites (mosquito allergy) is generated by mosquito bites, which cause strong topical reaction and exhibit not only formation of blisters, necrosis, ulceration and the like, but also systemic reactions such as fever, hepatosplenomegaly and lymphadenopathy, and the patients may sometimes have crusts after the formation of blisters. Therefore, particularly in the case where the symptom of hypersensitivity to mosquito bites is shown, the examination for whether chronic active EB virus infections is found or not is important for making the later treatment plan.

When virus enters and infects cells, their RNA and DNA of the virus itself are released in the infected cells and the virus replicates. Though the infected cells are controlled by RNA and DNA of the virus and usually killed, newly replicated viruses are released before the death of the cells, and infect other cells to spread. Some viruses do not kill the cells, but change its function to make normal cell division uncontrollable and cause the cells to become cancerous, and some viruses direct their DNA and RNA to invade into a host cell and be dormant (latent infections) but proliferate again once the cell gets injured.

In latent infections, when the immunological reaction of the host is maintained, EB virus controls the expression of viral antigen and expresses minimum functional molecule to maintain a state of latent infections, thus taking the form of infections as if the EB virus avoids an immunological reaction by host (“Nippon Iji Shinpo (Japan Medical Journal)”, No. 4136, p. 33-36 (2003)). Viral gene products expressed from episomal genome characteristic to latent infections are extremely limited. In case of EB virus, for example, proteins such as EBNA-1, EBNA-2, EBNA-3A, EBNA-3B, EBNA-3C, EBNA-LP; LMP-1, LMP-2A, and LMP-2B have been identified as a gene product related to latent infections, respectively. Further, EBER, or EB virus-encoded small RNA, BARTs (BARFO) and the like are identified as a gene product related to latent infections. Moreover, virus-derived mRNA which can be present in the cytoplasm of the infected cell can be identified as a gene product related to latent infections. Latent infections of EB virus are classified into three types of Latency I, II and III according to the presence or absence of the expressed protein described above. On the other hand, RNAs from EBER, BARFO and the like can be found in nucleus or cytoplasm in any fashion of latent infections. EBER1 and EBER2 have been known as EBER and, for example, EBER1 is present in large amounts in the nucleus of infected cell at 10⁶ to 10⁷ copies/cell. In the present invention, a gene product related to latent infections may be any described above, but RNA such as EBER or BARTs (BARFO), which can be present in nucleus or cytoplasm is preferred, and EBER1 which can be present in nucleus is more preferred.

In the present invention, crusts and/or scales formed on the skin are not limited to those directly formed by viral infections, but referred to any which are supposed to be caused by indirect viral infections such as hypersensitivity to mosquito bites described above. Herein, crusts have the same meaning as commonly used by those skilled in the art, thus it includes dried and solid effusion, secrete, blood and necrosis tissue covering the surface of erosion or ulcer. Further, scales have the same meaning as also commonly used by those skilled in the art, thus it is epidermal cuticle thickened and peeled. They correspond to so-called “scab”, consisting of a variety of cell components adhered to the surface of epidermal stratum corneum. The majority of the components forming crust are the stratum corneum of epidermal cells, and parts are infiltrating leukocytes present in blood such as lymphocytes and neutrophils. Complexes such as crusts and/or scales which are adhered on the skin surface will be used as a test sample, so noninvasive examination involving no pain can be realized.

In the present invention, a method for sampling crusts and scales is not limited in particular. For example, a sample can be collected by pressing the adhesive surface of an adhesive member to crusts and scales formed on the skin and peeling the adhesive member from the skin to entrap fragments of crusts and scales onto the adhesive surface. Further, the fragments of crusts and scales formed on the skin can be peeled and collected by a device such as tweezers or directly by hand and nails. Samples can be collected at any locations where no laboratory institution is available in the neighborhood, for example at home, schools, workplaces and hospital rooms by using the method described above.

In the present invention, any adhesive member may be used as long as it is adhesive, and they are not to be limited in particular, including adhesive sheets and adhesive tapes. In particular, cellophane tape and surgical tape may be mentioned, but not be limited to them, and any adhesive member which can collect crusts and/or scales formed on the skin in the present invention may be available. The collected test sample can be sealed, for example, by folding the adhesive member in two or by covering the adhesive layer with a protective sheet, and can be placed in a bag for exclusive use to prevent the sample from leaking outside and can be sent to a laboratory institution and the like.

The fragments peeled by tweezers, by hand or the like can also be placed in a special bag and be sent to a laboratory institution and the like. The collected test sample is preferably stored and sent under a dry condition.

Cells in crusts and scales are in a state of dry necrosis, so RNAs and the like contained in the cells are kept without degradation under a stable condition. Therefore, a gene product related to latent infections which may be present in the test sample collected by the method described above can be stored under a stable condition at a laboratory institution. In particular, as crusts in a lesion contain large amounts of virus-infected cells which are in a state of dry necrosis, host mRNA as well as a large amount of virus-related RNA present in nucleus are extractable, thus a sensitive and highly specific detection method can be provided.

The detection method for latent viral infections of the present invention comprises 1) collecting crusts and/or scales formed on the skin for a test sample, and in addition the method can further comprise the following steps:

2) extracting RNA or DNA of a gene product related to latent viral infections from the test sample;
3) amplifying nucleic acid based on the extracted RNA or DNA; and
4) detecting latent viral infections from the amplified nucleic acid.

The test sample is separated from the adhesive member through the use of, for example, tweezers and the like or is taken out of the sample storage bag, and then is placed into a nucleic acid extraction tube to extract nucleic acid. A well-known method per se can be used for extracting nucleic acid from the test sample. For example, extraction can be performed by a method described in a textbook way such as “Genetic Engineering Experiment Note” vol. 2 (YODOSHA CO., LTD.) or by using commercially available RNA and DNA extraction kits. Further, any nucleic acid extraction method which will be developed in the future can also be applied.

Nucleic acids can be amplified based on the extracted nucleic acids by a well-known method per se. Any method for amplifying nucleic acid, for example, polymerase chain reaction method (PCR method, Science, 230:1350-1354, 1985), NASBA method (Nucleic Acid Sequence Based Amplification Method, Nature, 350, 91-92, 1991), LAMP method (Japanese Published Patent Publication No. 2001-242169-A) and the like may be applied. Preferably, PCR method may be employed.

In the present invention, nucleic acid to be extracted from a test sample may be either RNA or DNA as described above, but preferably examination is performed with RNA. Although the presence of virus can be proved by the presence of its DNA, when virus infects latently and forms lesion, examination should preferably be performed on RNA.

Now, EB virus infections will be explained by way of illustration. Crusts in a lesion contain large amounts of EB virus-infected cells. As crusts in a lesion are in a state of dry necrosis, host mRNA as well as a large amount of virus-related RNA present in nucleus in the crust cell in a lesion can be extracted. To enhance sensitivity and specificity of the examination, nucleic acid derived from EB virus can be amplified using RT-PCR method which targets large amounts of EBER1s produced at the time of the latent infections of EB virus.

Well-known primers and reverse transcriptases can be used for producing cDNA from viral RNA. Further, for example, EBER1 derived from EB virus is a small RNA consisting of 167 bases, so it is hard to produce cDNA by a usual reverse transcription method. In such a case, cDNA may be produced efficiently by reverse transcription using an antisense primer for EBER1. As a result, even when only a very small number of EB virus-infected cells can be observed, the presence of infected cells can be proved by PCR method. In this way, when the former nucleic acid to be amplified is RNA, cDNA can be prepared by a well-known method per se and the nucleic acid can be amplified.

Commonly known primers for use in amplifying nucleic acid and probes for use in detection can also be applied as appropriate depending on the desired detection site of the desired virus. For example, primers and probes for use in amplification and detection of nucleic acid of EB virus may be, in particular, those described in Patent Document No. 2, Patent Document No. 4 and Japanese Published Patent Application No. 2005-58218-A, to which amplification and detection methods for nucleic acid can also be referred.

The present invention covers a detection method for latent viral infections and its kit for examination.

The kit for examination to detect virus of the present invention may comprise at least:

1) antisense oligonucleotide for reverse transcription of a gene product related to latent viral infections,
2) a primer set for amplifying a gene product related to latent viral infections, and
3) a primer set for amplifying a housekeeping gene.

In particular, preferably the kit comprises:

1) the oligonucleotide shown as SEQ ID NO: 1 in the sequence listing, 2) a primer set for amplifying EBER1, which is made up by combining the oligonucleotides shown as SEQ ID NOs: 2 and 1 in the sequence listing, and 3) a primer set for amplifying β2-microglobulin, which is made up by combining the oligonucleotides shown as SEQ ID NOs: 3 and 4 in the sequence listing.

In the present invention, β2-microglobulin is preferable as a housekeeping gene because it can be amplified by PCR under exactly the same condition as the amplifying condition of EBER1 from EB virus. In particular, MgCl2 is marked by a final concentration of 1.5±0.2 mM per 25 μl of the mixture of cDNA derived from EBER1 and cDNA derived from β2-microglobulin (B2-MG) for PCR operation, and further in PCR step, annealing is marked by setting the temperature in the range of 62±2° C.

Further, the kit for examination may comprise an adhesive member and a storage bag for test sample to collect crusts and/or scales appeared on the skin surface.

### EXAMPLES

The present invention will be explained by Examples below, and it will be clear that the present invention will not be limited by them.

### Example 1

### Identification of EBER1

### Extraction of RNA

1. Crusts were collected on cellophane tape from skin surface, and then the cellophane tape was folded in two to seal and store the crust.
2. The crusts sealed and stored in cellophane tape were placed into a tube for extracting nucleic acid with tweezers or the like, and 1 ml of TRIzol (GIBCO) was added thereto, and the cells were lysed by pipetting.
3. After the tube was left at room temperature for five minutes, 200 μl of chloroform was added and the tube was shaken vigorously for 15 seconds.
4. After left at room temperature for 2 to 3 minutes, the tube was centrifuged at 12,000 g for 15 minutes at 4° C.
5. 1 μl of glycogen (in concentration of 20 μg/μl) was placed into a new tube. After centrifugation described above, 600 μl of the supernatant was obtained, and to which the same amount of isopropanol was added and stirred.
6. The tube was left out at room temperature for 10 minutes, and RNA was extracted and then the tube was centrifuged at 12,000 g for 10 minutes at 4° C.
7. The supernatant was discarded, and 1 ml of 75% ethanol was added to the precipitated RNA pellet, and then the tube was moderately shaken and further centrifuged at 10,000 g for 5 minutes at 4° C. The supernatant was discarded again, and 1 ml of 75% ethanol was added to the RNA pellet and was centrifuged at 10,000 g for 5 minutes at 4° C.
8. The supernatant was discarded, and ethanol was evaporated and then the precipitate was dissolved in 20 μl of distilled water.
9. The concentration of RNA was measured by an absorption spectrometer and was diluted with distilled water such that the concentration was set to 0.1 μg/μl.

### Reverse Transcription (RT) Operation

1. 19.4 μl of Random hexamer (100 pmol/μl) (produced by TAKARA BIO INC.) and 2.59 μl of EBER1 anti-sense primer (100 pmol/μl) were mixed with 78.01 μl of distilled water to prepare RT-primer mixture (primer mixture).
2. 3.875 μl of RT-primer mixture was added to the RNA solution (0.1 μg/μl) (2 μl), heated to 60° C. for 10 minutes and then cooled with ice.
3. The following reagents were added and reacted for an hour at 37° C.

5×RT buffer (produced by Invitrogen) 2 μl, final: 50 mM Tris (pH8.3), 75 mM KCl, 3 mM MgCl₂

100 mM DTT (produced by Invitrogen) 1 μl, final: 10 mM

10 mM dNTP (produced by Invitrogen) 0.5 μl, final: 0.5 mM

RNAsin (produced by Promega) 0.125 μl (5 u)

MMLV-RT (produced by Invitrogen) 0.5 μl (100 u)

4. The tube was heated to 95° C. for ten minutes to inactivate reverse transcriptase.
5. After dilution with 10 μl of distilled water, the mixture of EBER1 and β2-microglobulin cDNA (corresponded to RNA at a concentration of 0.01 μg/μl) was obtained and stored.
6. Antisense oligonucleotide for the reverse transcription of EBER1

| | |
|---|---|
| AS-C1 5′-AAAACATGCGGACCACCACC-3′ | (SEQ ID NO: 1) |

PCR Operation (Common to both EBER1 and β2-Microglobulin)

### 1. Preparation of Primer Mixture

5 μl of each sense primer (100 pmol/μl) and 5 μl of each anti-sense primer (100 pmol/μl) for both EBER1 and β2-microglobulin were mixed to prepare totally 20 μl of a primer mixture.

### 2. PCR Primer

### For EBER1:

| | | |
|---|---|---|
| S-C1 | 5′-AGGACCTACGCTGCCCTAGA-3′ | (SEQ ID NO: 2) |
| AS-C1 | 5′-AAAACATGCGGACCACCAGC-3′ | (SEQ ID NO: 1) |

### For β2-Microglobulin:

(SEQ ID NO: 3)

| | |
|---|---|
| S-B2-MG | 5′-TACATGTCTCGATCCCACTTAACTAT-3′ |

(SEQ ID NO: 4)

| | |
|---|---|
| AS-E2-MG | 5′-AGCGTACTCCAAAGATTCAGGTT-3′ |

3. The following reagents were added to 5 μl of cDNA mixture.

10× buffer (produced by Promega) 2.5 μl, (final: 20 mM Tris (pH8.4), 40 mM KCl)

25 mM MgCl₂ 1.5 μl, (final: 1.5 mM)

2.5 mM dNTP (produced by Invitrogen) 2 μl, (respectively final: 200 μM)

primer mixture 0.4 μl, (respectively final: 10 pmol)

Taq polymerase (produced by Promega) 0.25 μl (1.25 u)

distilled water, final vol: amount 25 μl

### 4. PCR Amplifying Conditions

| | |
|---|---|
| denaturation | 94° C., 45 seconds |
| annealing | 62° C., 30 seconds |
| extension | 72° C., for one minute |
| 30 cycles | performed |

### Verification

Verification was performed by the run on 2% agarose gel according to the method described below.

1. 2% agarose gel was made by dissolving agarose (produced by TAKARA BIO INC.) in 1×TAE buffer (Tris 0.04 M, glacial acetic acid 0.04 M, 0.5 M EDTA (pH8.0) 0.001 M) into a concentration of 2%, adding ethidium bromide solution (10 mg/ml) at a rate of 1 μl per 100 ml of gel solution and mixing well.
2. 5 μl of PCR product was mixed with 1 μl of 6× Loading dye (produced by TOYOBO) and the solution was injected into each well. Electrophoresis was performed approximately for 30 minutes at a voltage of 100V, UV emitted, and the presence of gel band was checked. If RNA was extracted, a band can be seen at 295 bp as β2-microglobulin. Further, if EB virus is present, a band can be seen at 166 bp as EBER1.

### Result

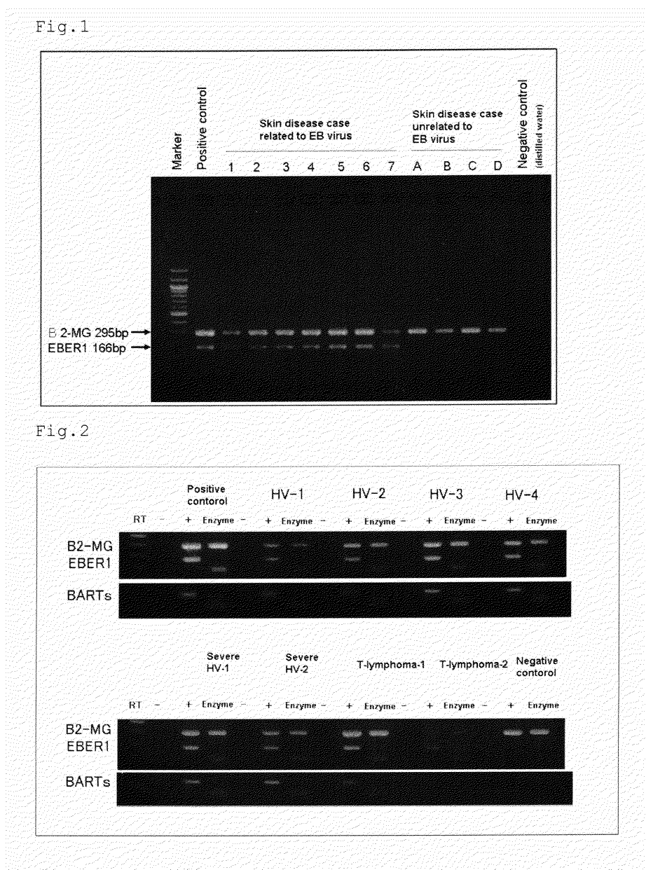

As shown in FIG. 1, two bands were found at 295 bp (β2-microglobulin) and 166 bp (EBER1) in positive control (using EB virus-infected cell strain), and the extraction of RNA was verified, which further proved the presence of EB virus-infected cell.

Likewise, the presence of EB virus-infected cell was proved in all cases of skin disease related to EB virus (hydroa vacciniforme and NK/T-cell lymphoma) from 1 to 7.

On the other hand, in the case of skin diseases unrelated to EB virus, the band of EBER1 (166 bp) was not observed from A to D, thereby showing no EB virus-infected cells. Further, the same method was applied to 12 cases of skin diseases unrelated to EB virus except for A to D, which showed no EB virus-infected cells.

### Example 2

### Verification of BARTs (BARF0)

RNA was extracted in the same manner as in Example 1 and reverse transcription was performed.

### PCR Operation for BARTs (BARF0)

### 1. Preparation of Primer Mixture

Verification of BARTs (BARF0) was performed by nested RT-PCR operation using an outer and an inner primer sets. First, in outer primer set, 5 μl of sense primer (100 pmol/μl) and 5 μl of anti-sense primer (100 pmol/μl) were mixed to prepare totally 20 μl of primer mixture. Likewise, in an inner primer set, 5 μl of sense primer (100 pmol/μl) and 5 μl of anti-sense primer (100 pmol/μl) were mixed to prepare totally 20 μl of primer mixture.

### 2. PCR Primer

### Outer Primer Set:

(SEQ ID NO: 5)

| | |
|---|---|
| BARTs-VB-S: | 5′-TGAGGGAAATAACCAGGATCACCA-3′ |

(SEQ ID NO: 6)

| | |
|---|---|
| BARTs-VIIA-AS: | 5′-GCTTCTCCTCGGACATCCAGT-3′ |

### Inner Primer Set:

(SEQ ID NO: 3)

| | |
|---|---|
| BARTs-VB-II-S: | 5′-TGAAGAAGGAGATGAAACCAGAGACCA-3′ |

(SEQ ID NO: 8)

| | |
|---|---|
| BARTs-VI-AS: | 5′-GACGAACAGCGTGCCTCCAA-3′ |

The reagents to be added to cDNA and the denaturation and extension conditions in PCR amplification were the same as in Example 1, and annealing was performed at 62° C. when the outer primer was used and at 64° C. when the inner primer was used. By nested RT-PCR operation, 28-cycle amplification was performed using the outer primer set, and then 23-cycle amplification was performed using the inner primer set.

Verification was performed by electrophoresis and the band checking was done in the same manner as in Example 1 and the results were shown in FIG. 2.

As shown in FIG. 2, two bands were observed at bp (32-microglobulin) and 166 bp (EBER1) and another band at 142 bp (BARTs) in positive control (using EB virus-infected cell line), thus being confirmed that RNA was extracted, which proved the presence of EB virus-infected cell.

Likewise, the presence of EB virus-infected cells was observed in skin disease cases related to EB viruses (HV-1 to 4, Severe HV-1, 2 and T lymphoma-1).

On the other hand, the bands of EBER1 and BARTs (BARF0) were not observed in negative control, which proved no EB virus-infected cell.

The “enzyme” in the figure is those treated with specific restriction enzymes (SmaI and NaeI (TOYOBO)) contained in the genetic sequences of the amplified products of EBER1 and BARTs (BARF0) and is subjected to electrophoresis. Bands were observed at 65 bp and 88 bp in EBER1 and at 64 bp and 78 bp in BARTs (BARF0). “RT−” means those using non-reverse-transcribed RNA in PCR and “RT+” means those using reverse-transcribed cDNA in PCR.

### Experimental Example 1

Crusts were collected from 15 patients suffering from diseases related to EBV and 52 patients suffering from diseases unrelated to EBV, and were examined on EBER1 and BARTs (BARF0) by the method described above, and the results were shown in Tables 1 and 2. It was judged that when both results were positive, latent infections of EBV was present, and when both results were negative or either result was shown positive, latent infections of EBV was absent. In that case, examination sensitivity was 93.3%, specificity 100%, positive likelihood ratio infinite and negative likelihood ratio 0.067. Thus, it can be believed that the method by collecting crusts and examining on EBER1 and BARTs (BARF0) is extremely excellent.

TABLE 1

| | EBER1 | | BARTs | |
|---|---|---|---|---|
| | Positive | Negative | Positive | Negative |
| | case | case | case | case |
| Diseases related to EBV | 15 | 0 | 14 | 1 |
| (15 cases) | | | | |
| Diseases unrelated to EBV | 4 | 48 | 4 | 48 |
| (52 cases) | | | | |

| | | |
|---|---|---|
| Sensitivity | 100% | 93.3% |
| Specificity | 92.3% | 92.3% |
| Positive likelihood ratio | 12.9 | 12.1 |
| Negative likelihood ratio | 0 | 0.0073 |

TABLE 2

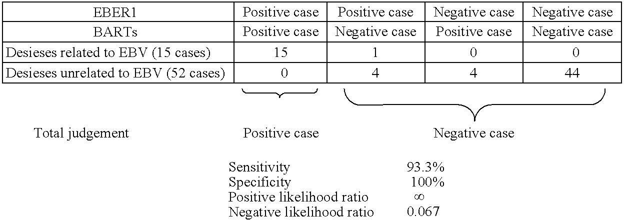

### INDUSTRIAL APPLICABILITY

As explained above, the detection method of the present invention can examine latent viral infections of, for example, EB virus using crusts and/or scales as a test sample. Diseases caused by latent infections of EB virus such as hydroa vacciniforme, hypersensitivity to mosquito bites and chronic active EB virus infections are often found in children, so collecting test sample involving pain and invasion should be avoided as much as possible. Crusts and/or scales on the skin surface can be collected at home, which means that test samples can be collected without pain and invasion, and sent to a laboratory institution at a distant place. Crusts and/or scales contain dry necrosed cells, so when latently infected with EB virus, the test sample contains large amounts of gene products related to latent infections such as EBER1 and BARTs (BARF0), thus they are stabile. Therefore, the detection method of the present invention comprising a step of amplifying nucleic acid using crusts and/or scales as a test sample is highly specific to gene products related to latent infections, thus providing a great contribution as a clinical examination. Further, a kit for examination using in the detection method of the present invention is also really useful.

### SEQUENCE LISTING

GPO 6-1011.ST25.txt

1. (canceled)

2. The detection method for latent viral infections, wherein a gene product related to latent infections which may be present in test sample are detected, wherein the method comprises:
1) collecting crusts and/or scales formed on the skin for a test sample,
2) extracting RNA which is a gene product related to latent viral infections from the test sample,
3) amplifying nucleic acid based on the extracted RNA, and
4) detecting a product generated by latent viral infections in the amplified nucleic acid.

3. The detection method according to claim 2, wherein the virus is EB virus (Epstein-Barr virus) or KSHV (Kaposi's sarcoma-associated herpes virus).

4. The detection method according to claim 3, wherein the gene product related to latent viral infections is RNA which can be present in the nucleus or cytoplasm of infected cells.

5. The detection method according to claim 4, wherein RNA which can be present in the nucleus or cytoplasm of the infected cells is EB virus-encoded small RNA (EBER) derived from EB virus and/or BamHI A rightward transcripts (BARTs (BARFO)).

6. The detection method according to claim 5, wherein the step of amplifying nucleic acid comprises a step of simultaneously amplifying EBER-derived acid and β2-microglobulin-derived nucleic acid under the same amplifying condition as the above.

7-12. (canceled)

13. The detection method according to claim 6, wherein the oligonucleotides shown as SEQ ID NO: 2 (5′-AGGACCTACGCTGCCCTAGA-3′) and SEQ ID NO: 1 (5′-AAAACATGCCGACCACCAG C-3′) in the sequence listing are used as a primer set for amplifying EBER-derived nucleic acid, and the oligonucleotides shown as SEQ ID NO: 3 (5′-TACATGT CTCGATCCCACTTAACTAT-3′) and SEQ ID NO: 4 (5′-AGCGTACTCCAAAGATTCAGGTT-3′) in the sequence listing are used as a primer set for amplifying β2-microglobulin-derived nucleic acid.

14. The detection method according to claim 6, wherein the step of amplifying nucleic acid further comprises a step of amplifying nucleic acid derived from BARTs (BARF0).

15. The detection method according to claim 14, wherein the oligonucleotides shown as both SEQ ID NO: 5 (5′-TGAGGGAAATAACCAGGATCACCA-3′) and SEQ ID NO: 6 (5′-GCTTCTCC TCGGACATCCAGT-3′) and both SEQ ID NO: 7 (5′-TCAAGAAGGAGATGAAACCAGAGACCA-3′) and SEQ ID NO: 8 (5′-GACGAACAGCGTGCCTCCAA-3′) in the sequence listing are used as primer sets for amplifying nucleic acid derived from BARTs (BARF0).

16. The detection method according to claim 6, wherein the step of amplifying nucleic acid is performed by polymerase chain reaction (PCR) operation and a final concentration of MgCl₂ is 1.5±0.2 in M per 25 μl of cDNA mixture for PCR operation.

17. The detection method according to claim 16, wherein annealing is performed at temperature in the range of 62±2° C. and/or 64±2° C. in the PCR operation.

18. A kit for examination using the detection method according to claim 2, comprising at least the following 1) to 3):
1) antisense oligonucleotide for reverse transcription of a gene product related to latent viral infections,
2) a primer set for amplifying a gene product related to latent viral infections,
3) a primer set for amplifying a housekeeping gene.

19. The kit for examination according to claim 18, wherein the antisense oligonucleotide and/or the primer set in said 1) to 3) are the following:
1) the oligonucleotide shown as SEQ ID NO: 1 (5′-AAAACATGCGGACCACCAGC-3′) in the sequence listing,
2) a primer set for amplifying EBER-derived nucleic acid, wherein the primer set is made up by combining the oligonucleotides shown as SEQ ID NO: 2 (5′-AGGACCTACGCTGCCCTAGA-3′) and SEQ ID NO: 1 (5′-AAAACATGCGGACCACCAGC-3′) in the sequence listing,
3) a primer set for amplifying β2-microglobulin-derived nucleic acid, wherein the primer set is made up by combining the oligonucleotides shown as SEQ ID NO: 3 (5′-TACATGTCTCGATCCCACTTAACTAT-3′) and SEQ ID NO: 4 (5′-AGCGTACTCCAAAGATTCAGGTT-3′) in the sequence listing.

20. The kit for examination according to claim 19, further comprising a primer set for amplifying nucleic acid derived from BARTs (BARF0), wherein the primer set is made up by combining the oligonucleotides shown as both SEQ ID NO: 5 (5′-TGAGGGAAATAACCAGGATCACCA-3′) and SEQ ID NO: 6 (5′-GCTTCTCCTCGGACATCCAGT-3′) and both SEQ ID NO: 7 (5′-TGAAGAAGGAGATGAAACCAGAGACCA-3′) and SEQ ID NO: 8 (5′-GACGAACAGCGTGCCTCCAA-3′) in the sequence listing.
